(19) United States
(12) Patent Application Publication
(10) Pub. No.: US 2021/0230155 A1
(43) Pub. Date: Jul. 29, 2021

(54) PROCESS FOR PREPARATION OF ((3R,11BR)-1,3,4,6,7,11B-HEXAHYDRO-9,10-DI(METHOXY-D3)-3-(2-METHYLPROPYL)-2H-BENZO[A]QUINOLIZIN-2-ONE

(71) Applicant: Hetero Labs Limited, Hyderabad (IN)

(72) Inventors: Bandi PARTHASARADHI REDDY, Hyderabad (IN); Kura RATHNAKAR REDDY, Hyderabad (IN); Bandi VAMSI KRISHNA, Hyderabad (IN); Maruthi JANAKI RAM REDDY, Hyderabad (IN); Gutta MADHUSUDHAN, Hyderabad (IN)

(21) Appl. No.: 17/050,862

(22) PCT Filed: Apr. 25, 2019
(86) PCT No.: PCT/IB2019/053411

§ 371 Date: Oct. 27, 2020

(30) Foreign Application Priority Data
Apr. 27, 2018 (IN) 201841015985

(51) Int. Cl. C07D 455/06

(52) U.S. Cl. CPC C07D 455/06; C07B 2200/05

(57) ABSTRACT

The present invention provides a process for preparation of deutetrabenazine using tetrabenazine as starting material.

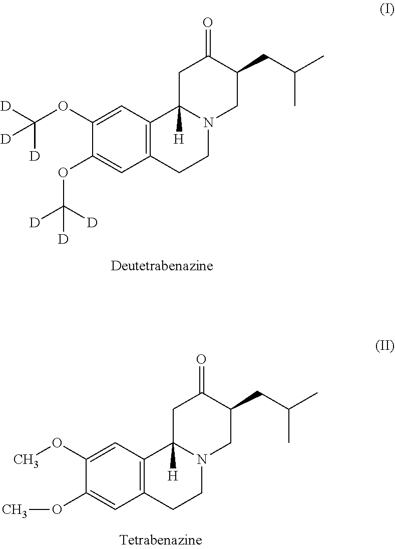

### FIELD OF INVENTION

The present invention provides a process for preparation of Deutetrabenazine using novel intermediates.

### BACKGROUND OF THE INVENTION

Deutetrabenazine having the chemical name ((3R,11bR)-1,3,4,6,7,11b-Hexahydro-9,10-di(methoxy-d3)-3-(2-methylpropyl)-2H-benzo[a]quinolizin-2-one and the structural formula I.

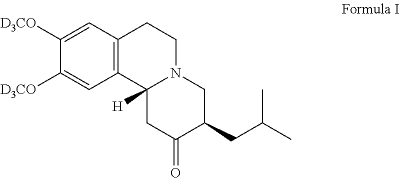

Deutetrabenazine was first deuterated compound approved by the FDA, marketed under the brand name Austedo® by Teva, for the treatment of Huntington's disease. Chemically Deutetrabenazine is an analog of an approved drug Tetrabenzine in which the hydrogen atoms at the 9- and 10-methoxy (—OCH₃) substituents of Tetrabenzine are replaced by deuterium. Both Tetrabenzine and Deutetrabenazine are racemic mixtures. Tetrabenazine is rapidly metabolized in vivo to α- and β-isomers of dihydrotetrabenazine, which provide systemic exposure of pharmacological activity. Further α- and β-isomers of dihydrotetrabenazine by CYP2D6 to the 9- and 10-desmethyl metabolites results in large fluctuations in plasma concentrations and the need for frequent dosing.

U.S. Pat. No. 8,524,733 B2 discloses Deutetrabenazine for the first time. This patent also discloses a process for preparing Deutetrabenazine, which is as shown below:

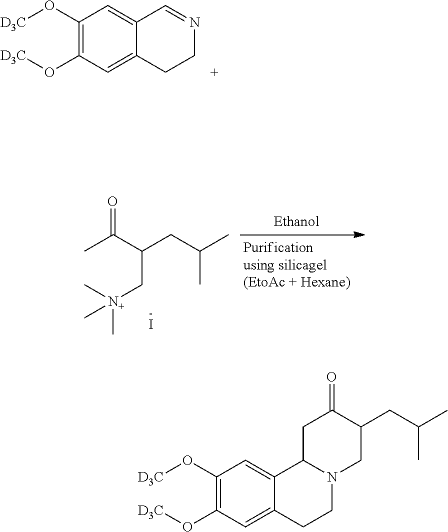

As per this process, the present inventors observed that the process is not suitable for commercial scale due to use of column chromatography techniques for the isolation and purification of duetetrabenazine.

US 2015/0152099 A1 discloses another process for preparation of Deutetrabenazine, which is as shown below:

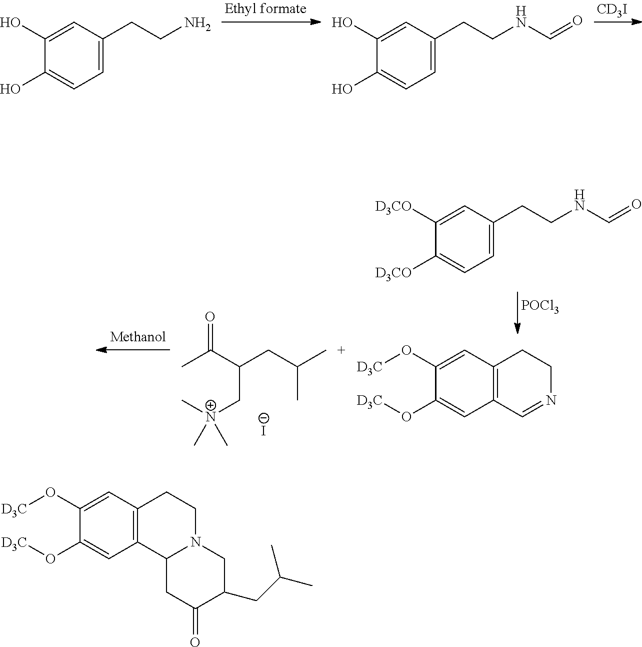

US 2016/0030414 A disclose another process for preparation of Deutetrabenazine, which is as shown below:

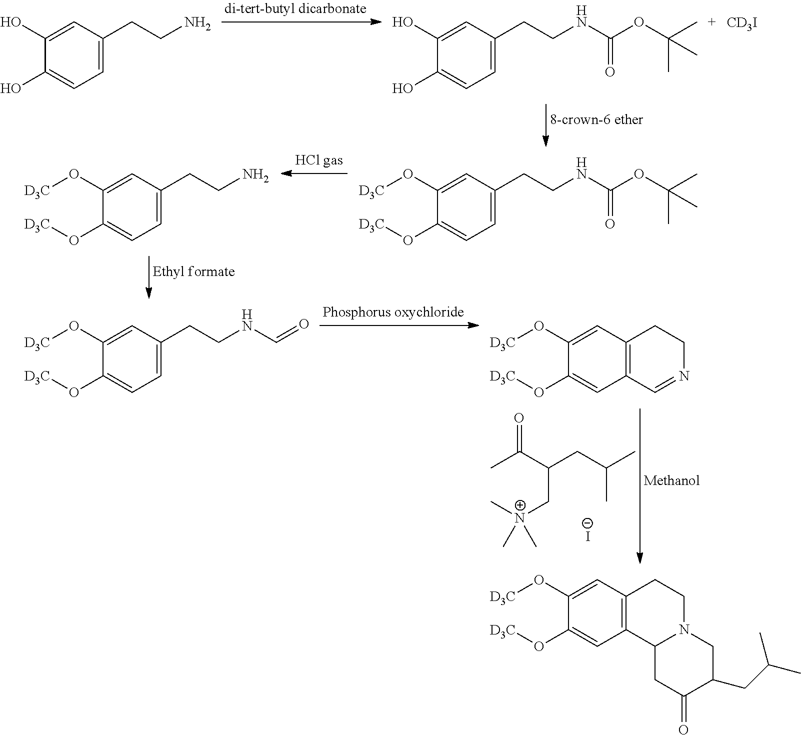

As per this process, the present inventors observed that use of deuterated methyl iodide at the early stage of reaction. However note that deuterated methyl iodide is costly and also obtained product in low yields in this process due to reasons which are using of expensive reagents in early stages not suitable commercially, economically and industrially.

The prior-arts have been provided different processes for preparing Deutetrabenazine and its intermediates, however the present inventors provides a different process for preparation of Deutetrabenazine.

### Objectives

The objective of the present invention is to provide a process for the preparation of Deutetrabenazine having high yields and high purity.

Another objective of the present invention is to provide novel intermediates, which are useful for preparation of Deutetrabenazine

### Summary of the Invention

A process for the preparation of Deutetrabenazine compound of formula I,

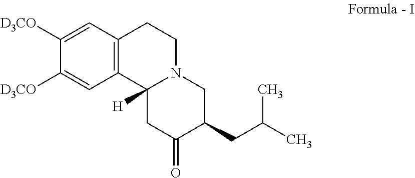

which comprises the following steps:
i. demethylation of Tetrabenazine of compound of Formula II,

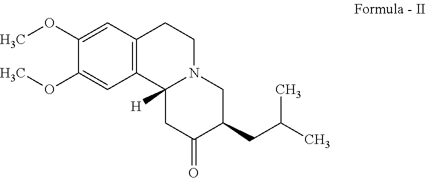 [SMILES: COc1cc2c(cc1OC)[C@H]1CC(=O)[C@H](CC(C)C)CN1CC2]

with an acid to yield di-hydroxy benzoquinoline compound of formula III or a salt thereof;

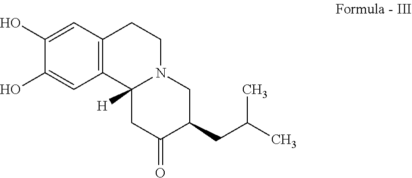 [SMILES: CC(C)C[C@@H]1CN2CCc3cc(O)c(O)cc3[C@H]2CC1=O]

ii. optionally protecting the keto group of di-hydroxy benzoquinoline compound of Formula III with protecting group to yield keto protected di-hydroxy benzoquinoline compound of Formula IV;

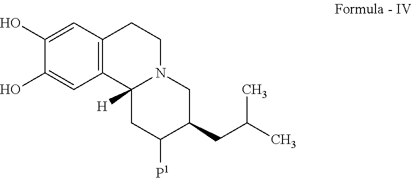

wherein P¹ is protecting group
iii. reacting the di-hydroxy benzoquinoline compound of formula III or keto protected di-hydroxy benzoquinoline compound of Formula IV with deuterium source to yield Deutetrabenzine compound of formula I or keto protected deutetrabenazine compound of Formula V

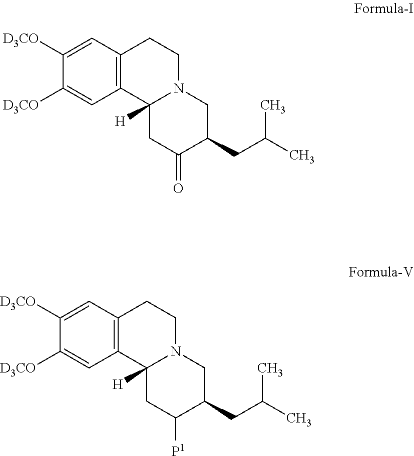

iv. optionally de-protecting the keto protected deutetrabenazine compound of Formula V using an acid to yield Deutetrabenazine compound of formula I,

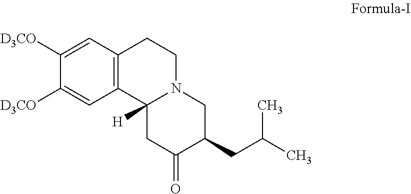

v. optionally purifying the obtained deutetrabenazine compound of formula I and
vi. isolating the obtained deutetrabenazine compound of formula I

The present invention is also relates to novel intermediates compound of formula IV and compound of formula V which are useful in the process for preparation of Deutetrabenazine compound of formula I.

### Compound of Formula IV;

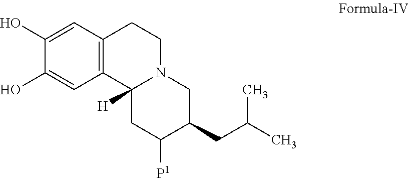

### Compound of Formula V

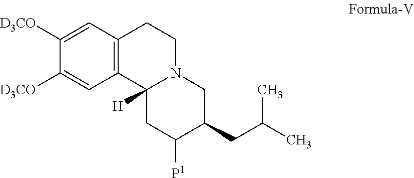

### DETAILED DESCRIPTION OF THE INVENTION

The present invention relates to a process for the preparation of deutetrabenazine using tetrabenazine as a starting material. Demethylating the Tetrabenazine compound of formula II in the presence of an acid wherein acid is selected form concentrated or aqueous Hydrobromic acid, acetic acid, sulfuric acid or mixture thereof to obtain di-hydroxy isoquinoline compound of formula III.

Another embodiment of the present invention is to provide a process for the preparation of keto protected dihydroxy benzoquinoline compound of Formula IV by reacting the di-hydroxy isoquinoline compound of formula III with protecting group. The protecting groups are selected from the group consisting of ethylene glycol, Isobutylene glycol, 1,3-Propylene glycol, 2,2-Dimethyl-1,3-propanediol; 1,3-Propanedithiol, 3-(Phenylsulfonyl)-1,2-propanedio. The reaction is carried out in presence of a solvent or mixture of solvents and an acid. The solvent can be selected from alcohols and ketones; wherein alcohols are selected from the group consisting of methanol, ethanol, 1-propanol, isopropanol; wherein ketones are selected from the group consisting of acetone, methyl isobutyl ketone, methyl isopropyl ketone. The acid can be selected form hydrochloric acid, hydro bromic acid; preferably hydrochloric acid in 1,4-dioxane. The reaction cab be carried out at 0 to 50° C.

Another embodiment of the present invention is to provide a process for preparation of keto protected deutetrabenazine compound of Formula V by deuterated the hydroxyl groups of keto protected di-hydroxy isoquinoline compound of formula IV with a source of deuterium, wherein the source of deuterium is selected from group consisting of deuterated methanol, deuterated methyl iodide, deuterated methyl bromide, deuterated dimethyl sulfate and deuterated dimethyl carbonate. The reaction can be carried out in presence of catalyst selected di-isopropyl azodicarboxylate (DIAD) or Diethyl azodicarboxylate and tri phenyl phosphine. The reaction can be carried out in the presence of solvent or mixture solvent selected from halogenated hydrocarbons solvents like dichloromethane, chloroform, ethylene dichloride and carbon tetrachloride and ethers like tetrahydrofuran.

Another embodiment of the present invention is to provide a process for preparation of deutetrabenazine compound of Formula I from di-hydroxy isoquinoline compound of formula III by deuterated the hydroxyl groups of di-hydroxy isoquinoline compound of formula III with a source of deuterium to obtain deutetrabenazine compound of Formula I, wherein the source of deuterium is selected from group consisting of deuterated methanol, deuterated ethanol, deuterated acetone deuterated methyl iodide, deuterated methyl bromide, deuterated dimethyl sulfate and deuterated dimethyl carbonate. The reaction can be carried out in presence of catalyst selected diisopropyl azodicarboxylate (DIAD) or Diethyl azodicarboxylate and tri phenyl phosphine. The reaction can be carried out in presence of solvent or mixture solvent selected from halogenated hydrocarbons solvents like dichloromethane, chloroform, ethylene dichloride and carbon tetrachloride; alcoholic solvents like methanol, ethanol, isopropyl alcohol, nitrile solvent like acetonitrile; kenote solvent like acetone, methyl isobutyl ketone, methyl isopropyl ketone, ethers like tetrahydrofuran, diethyl ether, di-isopropyl ether; esters like methyl acetate, ethyl acetate, isopropyl acetate and other solvents selected from toluene, dimethyl formamide, dimethyl sulphoxide and water

Another embodiment of the present invention is to provide a process for preparation of deutetrabenazine compound of formula I from keto protected deutetrabenazine compound of Formula V by de-protection of keto protected group, wherein the de-protection is carried out in presence of acid and solvent. wherein acid is selected form sulfuric acid, hydrobromic acid and hydrochloric acid. The reaction can be carried out in absent or presence of solvent or mixture of solvents selected form alcohols and ethers; wherein alcohols are selected from the group consisting of methanol, ethanol, 1-propanol, isopropanol; wherein ether are selected from the group consisting of tetrahydrofuran, di-tert-butyl ether, diethyl ether, di-isopropyl ether, methyl tert-butyl ether. The reaction can be carried out at room temperature.

Another embodiment of the present invention is to provide a process for preparation of deutetrabenazine compound of formula I can be prepared directly from di-hydroxy isoquinoline compound of formula III or via protecting the di-hydroxy isoquinoline compound of formula III to from keto protected di-hydroxy isoquinoline compound of formula IV, deuterated the keto protected di-hydroxy isoquinoline compound of formula IV to from deuterated keto protected di-hydroxy isoquinoline compound of formula IV and deprotecting the deuterated keto protected di-hydroxy isoquinoline compound of formula V to form deutetrabenazine.

Another embodiment of the present invention is optionally protecting the di-hydroxy isoquinoline compound of formula III and optionally deprotecting the deuterated keto protected di-hydroxy isoquinoline compound of formula V.

Another embodiment of the present invention is to provide the intermediate compounds of compound of formula III, compound of formula IV and compound of formula V are may be isolated or in-situ process. if isolated can be isolated by techniques known in art like filtration, evaporation, concentration etc.

As per the present invention the process for preparation of Deutetrabenazine has been shown schematically as below:

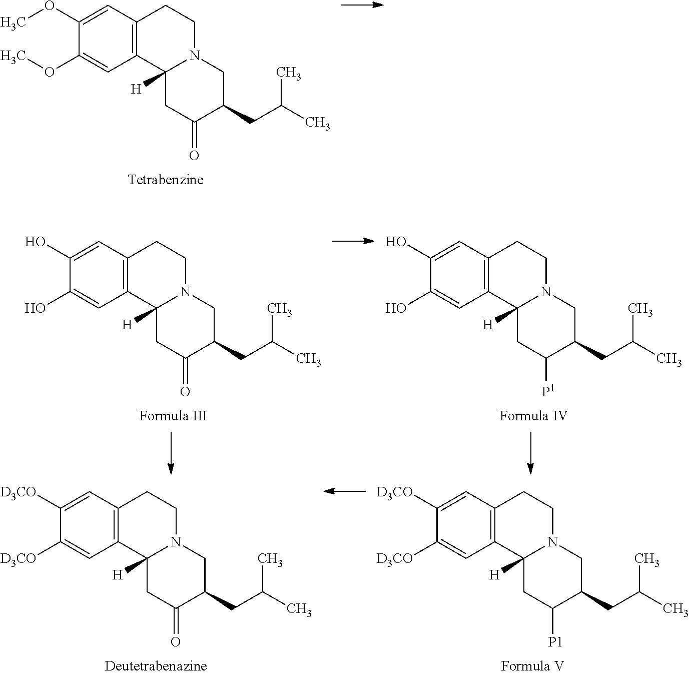

Another embodiment of the present invention provide a novel intermediates compound of formula IV and compound of formula V which are useful in preparation of Deutetrabenazine compound of formula I.

### Compound of Formula IV;

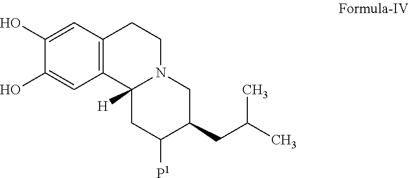

### Compound of Formula V

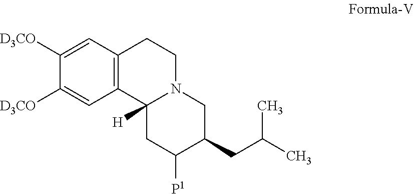

wherein P¹ is protecting group is selected from the group consisting of as follows

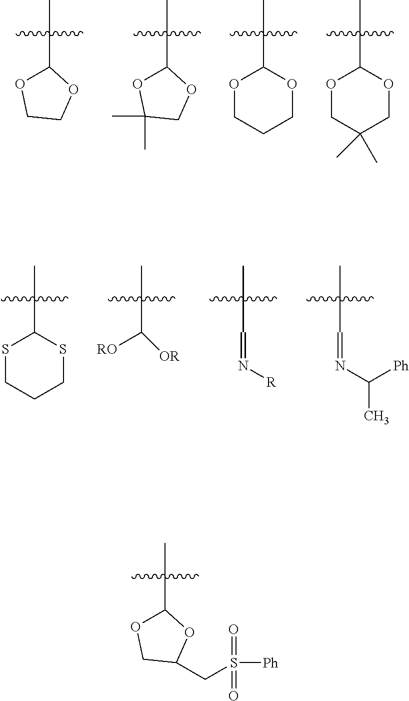

wherein R is selected from group consisting of alkyl or aryl.

Another embodiment of the present invention is to provide purification process of deutetrabenazine using solvents selected from alcoholic solvents selected from methanol, ethanol, isopropyl alcohol, ketones selected from acetone, methyl isobutyl ketone, methyl isopropyl ketone; nitrile selected from acetonitrile and other solvents selected from toluene, dimethyl formamide, dimethyl sulphoxide, hexane, heptane and water or mixture thereof.

The starting material Tetrabenazine compound of formula II can be prepared by method known in the prior-art.

The present invention is further illustrated by the following representative examples and does not limit the scope of the invention.

### EXAMPLES

### Example 1. Preparation of Di-Hydroxy Isoquinoline Compound of Formula III

Tetrabenazine (20 g) was dissolved in acetic acid (40 ml) and the reaction mixture was stirred. 33% aq. Hydrobromic acid in acetic acid (100 ml) was added and followed by aq. Hydrobromic acid (100 ml) was added to it. The reaction mass was heated to 115-120° C. for 4 to 6 hours. The reaction mass was concentrated under reduced pressure to remove excess Hydrobromic acid and Acetic acid. Di-isopropyl ether was added to the above solid and stirred for 30 minutes at 25-30° C. The solid was filtrate and concentrated under reduced pressure to give title compound.

### Yield: 21.6 g.

Chromatography purity by HPLC: 89.73%

### Example 2. Preparation of Di-Hydroxy Isoquinoline Compound of Formula III

Tetrabenazine (10 g) was dissolved in acetic acid (20 ml) at 25-30° C. 33% aq. Hydrobromic acid in acetic acid (60 ml) and 48% aq. Hydrobromic acid (30 ml) were added to it. The reaction mass was heated to 115-120° C. for 8 hours. The reaction mass was cooled to 50° C. and distil off the solvent under reduced pressure. Water was added to above residue and cooled to 10-15° C. The pH of reaction mass was adjusted to 7 to 7.5 with 40% aq. Sodium carbonate (100 ml) and extracted with ethyl acetate (3×100 ml) the ethyl acetate layer was washed with brine solution (100 ml) distill of the solvent under vacuum to obtained the till compound.

### Yield: 6 g.

Chromatography purity by HPLC: 96.5%

### Example 3. Preparation of Di-Hydroxy Isoquinoline Compound of Formula III

A mixture of Tetrabenazine (100 g), 48% aq. Hydrobromic acid (300 ml), 33% Hydrobromic acid in acetic acid (300 ml) are dissolved in acetic acid (300 ml) at 25-30° C. The reaction mixture was heated to 115-120° C. for 5-6 hours. The reaction mass was cooled to 30-35° C. and stirred for 45-60 minutes. Filter the solid and washed with water (300 ml). Water was added to above crude and pH was adjusted to 7.5-8.5 with aqueous ammonia solution and stirred for 30-45 minutes at 30-35° C. Filter the solid and washed with water (300 ml) and dried hot air oven at 55-60° C. for 3-4 hours to give title compound.

### Yield: 90 g.

Chromatography purity by HPLC: 96.98%

### Example 4. Preparation of Keto Protected Dihydroxy Benzoquinoline Compound of Formula IV

A mixture of di-hydroxy isoquinoline compound of formula III (21 g) and Methanol (105 ml) were stirred. Ethylene glycol (105 ml), Methyl tert-butyl ether (52 ml) and 4N hydrochlorid acid in 1,4-dioxane (100 ml) were added at 0° C. The reaction mixture was stirred at ambient temperature for 24 hours and concentrated under pressure at 35-40° C. The pH of reaction mass was adjusted to 7-8 with aq. Sodium bicarbonate at temperature 10-15° C. The reaction mass was extracted with methanol in dichloromethane (4×300 ml) and the organic extracts were washed with water and brine, dried (Na₂SO₄), solvent was evaporated under reduced pressure to obtained a pale yellow solid. The crude compound was added n-hexane (500 ml) and stirred for 30 minutes. The solid was filtrate and dried under reduced pressure to give title compound.

### Yield: 16 g.

Chromatography purity by HPLC: 94.12%

### Example 5. Preparation of Keto Protected Deutetrabenazine Compound of Formula V

A mixture of Triphenylphosphine (3.93 g), Diisopropyl azodicarboxylate (3.03 g) were dissolved in dichloromethane (16 ml) in nitrogen atmosphere and stirred. The reaction mass was cooled to 0° C. Deuterated methanol (2.2 ml) and keto protected dihydroxy benzoquinoline compound of Formula IV (2 g) were added to above reaction mixture and stirred at room temperature for 3 hours. The reaction mixture was quenched with ice water and extracted with ethyl acetate. The combined organic extracts were washed with water and brine, dried (Na₂SO₄), solvent was evaporated under reduced pressure to obtain a brownish yellow solid. The crude compound was added n-hexane (20 vol) and stirred for 1 hour. The solid was filtrate and dried under reduced pressure to give title compound.

### Yield: 1.3 g

Chromatography purity by HPLC: 88.92%

### Example 6. Preparation of Deutetrabenazine

keto protected deutetrabenazine compound of Formula V (1.3 g), was dissolved in methanol in tetrahydrofuran (1:1) at 0° C. and stirred. The reaction mixture was added 2M sulfuric acid solution drop wise and stirred for 8 hours at room temperature and concentrated under reduced pressure at 40° C. the reaction mixture was diluted with water (20 ml) and pH was adjusted to 7 to 8 with sodium bicarbonate solution and extracted with ethyl acetate and the organic extracts were washed with water and brine, dried (Na₂SO₄). The crude product was washed with 5 volumes of n-Hexane and filtered to obtain the title compound as off white sold.

### Yield: 1 g

Chromatography purity by HPLC: 98.73%

### Example 7. Preparation of Deutetrabenazine

Di-hydroxy isoquinoline compound of formula III (10 g) was dissolved in acetonitrile (150 ml) at 25-30° C. 18-crwon-6 ether (1.82 g), Deuterated iodomethane (15 g) and cesium carbonate (33.7 g) were added to above reaction mixture and heated to 40° C. for 1 hour. Cooled to 25-30° C. The reaction mixture was filtered and washed with acetonitrile the filtrate was distill off under vacuum at 40° C. and water was added to the obtained residue. The reaction mass was extracted with ethyl acetate and the ethyl acetate layer washed with 10% brine solution and distil off the solvent under vacuum to obtain the title compound.

### Yield: 6 g.

Chromatography purity by HPLC: 98.94%

### Example 8. Preparation of Deutetrabenazine

Di-hydroxy benzoquinoline compound of formula III (80 g) was dissolved in Toluene (320 ml) at 30-35° C. deuteriated methanol (45 ml) and triphenylphosphine (218 g) were added to above reaction mixture. a solution of diisopropylazodicraboxylate (DIAD) (165 mL) was added to above reaction mixture for 60-90 minutes and stirred for 1.5 hours to 2 hours at 45-50° C. Cooled to 3 0±5° C. The mixture was filtered and the filtrate was washed with toluene (80 ml), Sodium bisulfate (30 g) and water (300 ml) were added at 30-35° C. and stirred for 30-45 minutes. The aqueous layers were collected together, and the pH was adjusted to 9-10 using aqueous ammonia solution and stirred for 30-45 minutes at 30-35° C. Filtered the solid and wash with purified water (240 ml). isopropanol (160 ml) was added to the residue, and the mixture was heated to 80-85° C. and stirred for 30-40 minutes. The reaction mass was cooled to 0-5° C. and stirred for 1-1.5 hours. The solid was filtered, washed with isopropanol (40 ml), and dried hot air oven at 50-5° C. for 3-4 hours to give title compound.

### Yield: 65 g.

Chromatography purity by HPLC: 99.48%

### Example 9: Purification of Deutetrabenazine

Deutetrabenzine (60 g) was dissolved in acetone (300 ml) at 30-35° C. The reaction mixture was heated to at 55-60° C. and activated carbon was added to the above reaction mixture and stirred for 15-20 minutes at the same temperature. Filter the reaction mixture through hyflow bed and wash with acetone. The filtrate was hated at 55-60° C. and water was added and stirred for 30-45 minutes at the same temperature. Cooled to 0-5° C. and stirred for 1 hour to 1.5 hours. Filter the solid and washed with mixture of acetone and water and dried hot air oven at 50-5° C. for 3-4 hours to give title compound.

### Yield: 55 g.

Chromatography purity by HPLC: 99.86%

1. A process for the preparation of Deutetrabenazine compound of formula I, which comprises the following steps: 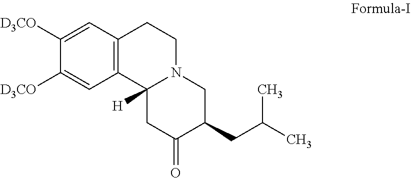
i. demethylation of Tetrabenazine of compound of Formula II, 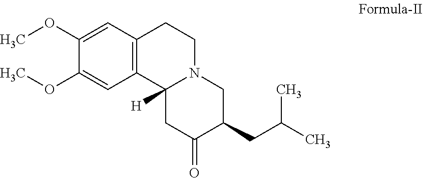 [SMILES: COc1cc2c(cc1OC)[C@H]1CC(=O)[C@H](CC(C)C)CN1CC2]
with an acid or mixture to yield di-hydroxy benzoquinoline compound of formula III or a salt thereof; 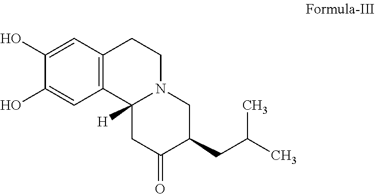 [SMILES: CC(C)C[C@@H]1CN2CCc3cc(O)c(O)cc3[C@H]2CC1=O]
ii. optionally protecting the keto group of di-hydroxy benzoquinoline compound of Formula III with protecting group to yield keto protected di-hydroxy benzoquinoline compound of Formula IV; 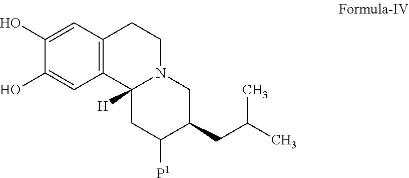
wherein P¹ is protecting group
iii. reacting the di-hydroxy benzoquinoline compound of formula III or keto protected di-hydroxy benzoquinoline compound of Formula IV with deuterium source to yield Deutetrabenzine compound of formula I or keto protected deutetrabenazine compound of Formula V 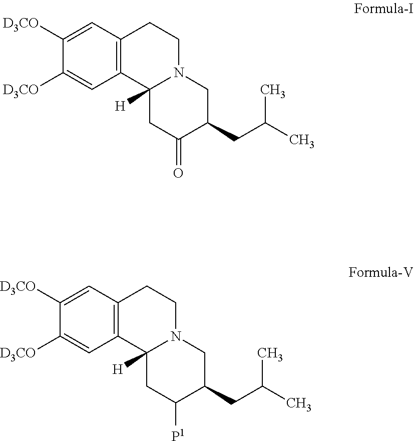
iv. optionally de-protecting the keto protected deutetrabenazine compound of Formula V using an acid to yield Deutetrabenazine compound of formula I, 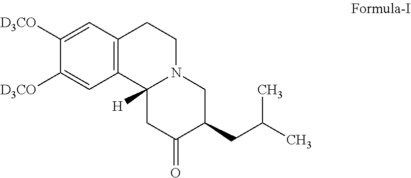
v. optionally purifying the obtained deutetrabenazine compound of formula I and
vi. isolating the obtained deutetrabenazine compound of formula I

2. The process as claimed in claim 1, where in step (i) an acid is selected from the group consisting of concentrated or aqueous hydrobromic acid, acetic acid, sulfuric acid or mixture thereof.

3. The process as claimed in claim 1, where in step (ii) the protecting groups are selected from the group consisting of ethylene glycol, Isobutylene glycol, 1,3-Propylene glycol, 2,2-Dimethyl-1,3-propanediol, 1,3-Propanedithiol, 3-(Phenylsulfonyl)-1,2-propanediol.

4. The process as claimed in claim 1, where in step (iii), the deuterium source is selected from group consisting of deuterated methanol, deuterated ethanol, deuterated acetone deuterated methyl iodide, deuterated methyl bromide, deuterated dimethyl sulfate and deuterated dimethyl carbonate.

5. The process as claimed in claim 1, where in step iii is carried out in presence of a catalyst and solvent.

6. The process as claimed in claim 5, wherein the catalyst is selected from group consisting of diisopropyl azodicarboxylate and diethyl azodicarboxylate.

7. The process as claimed in claim 1, wherein solvent is selected from halogenated hydrocarbons solvents like dichloromethane, chloroform, ethylene dichloride and carbon tetrachloride; alcoholic solvents like methanol, ethanol, isopropyl alcohol, nitrile solvent like acetonitrile; kenote solvent like acetone, methyl isobutyl ketone, methyl isopropyl ketone. ethers like tetrahydrofuran, diethyl ether, diisopropyl ether; esters like methyl acetate, ethyl acetate, isopropyl acetate and other solvents selected from toluene, dimethyl formamide, dimethyl sulphoxide and water.

8. The process as claimed in claim 1, wherein step (iv) an acid is selected from the group consisting of hydrobromic acid, hydrochloric acid or sulfuric acid.

9. The intermediate compounds of Formula IV and Formula V 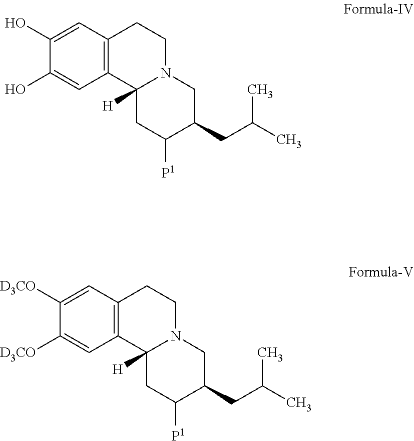
wherein P¹ is protecting group is selected from the group consisting of: 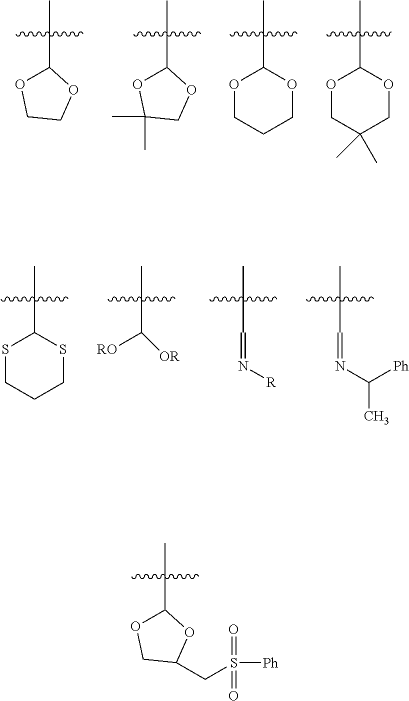
wherein R is selected from group consisting of alkyl or aryl.
